(19) United States
(12) Patent Application Publication
(10) Pub. No.: US 2009/0099459 A1
(43) Pub. Date: Apr. 16, 2009

(54) Device, System And Method For Determining The Effect Of Photodynamic Or Photothermal Tumor Therapy

(76) Inventors: Sune Svanberg, Lund (SE); Katarina Svanberg, Lund (SE)

Correspondence Address: INSKEEP INTELLECTUAL PROPERTY GROUP, INC, 2281 W. 190TH STREET, SUITE 200, TORRANCE, CA, 90504, US

(21) Appl. No.: 11/911,106

(22) PCT Filed: May 12, 2006
(86) PCT No.: PCT/SE2006/050121

§ 371 Date: Oct. 15, 2008

Related U.S. Application Data
(60) Provisional application No. 60/680,778, filed on May 13, 2005.

(30) Foreign Application Priority Data
May 12, 2005 (SE) 0501078-0

(51) Int. Cl. A61B 6/00

(52) U.S. Cl. 600/478

(57) ABSTRACT

A device and method for determining the effect of therapy in a tumour tissue of a human or a mammal under interstitial photodynamic or photothermal therapy by analyzing a liquid flow in a tissue of a human or a mammal. A first fibre is interstitially inserted in a first position of said tissue and connected to a light source, and a second fibre is interstitially inserted in a second position of said tissue for receiving light emitted from the first fibre. A detector is arranged for receiving the light from said second fibre for producing an output signal. An analysator receives the output signal from the detector and determines if there is a frequency component in the frequency area below about 1 MHz in the output signal, which is indicative of blood cell movement in the tissue. If the frequency component is below a threshold value, it is determined that there is no blood flow. In photothermal therapy, no blood flow is interpreted as the fact that the blood has coagulated and the therapy may be finalized. In photodynamic tumour therapy, changes in blood flow may be used to evaluate the treatment progression.

### RELATED APPLICATIONS

This application claims priority to International Patent Application No. PCT/SE2006/050121 filed May 12, 2006 entitled “A Device, System And Method For Determining The Effect Of Photodynamic Or Photothermal Tumor Therapy,” which claims priority to Swedish patent application No. 0501078-0 filed May 12, 2005 entitled “A Device For Analyzing A Liquid Flow” and U.S. Provisional Application Ser. No. 60/680,788 filed May 13, 2005 entitled “A Device For Analyzing A Liquid Flow,” all of which are hereby incorporated by reference.

### FIELD OF THE INVENTION

The present invention relates to a device, system and method for determining the effect of photodynamic or photothermal tumour therapy by interstitially analyzing blood flow in a tissue exposed to such therapy.

### BACKGROUND OF THE INVENTION

A system and method for therapy and diagnosis using interactive interstitial photodynamic tumour therapy and/or photothermal tumour therapy is disclosed in for example WO 03/041575 of same applicant as the present application. The system comprises several optical fibres inserted at selected positions in the tissue to be treated and/or diagnosed, such as cancer tissue.

For diagnosis, a laser light source may be connected to one of the fibres operating as transmission fibre and the other fibres operating as receivers for light passing through the tissue to the other fibres and further on to detectors. Each fibre is used in sequence for being a transmitter and the other fibres being receivers. By calculations, a tomographic image of certain properties of the tissue may be obtained, such as light flux or fluorescence properties arising from sensitizers.

For therapy, the same system may be used whereby light is transmitted through the fibres to the tissue for activation of sensitizers, which are localized to the tumour cells in advance. By means of the therapy, the cells are eradicated and the tissue goes into necrosis. Moreover, the system may be used for photo-thermal therapy comprising transmitting laser light through the fibres, which heats the tissue sufficiently for killing the tumour cells. It may be noted that tumour cells may be more sensitive to heat than other cells.

During use of this system, the signals received during such diagnosis as mentioned above comprise a signal component having a frequency around 104 Hz and with a varying spectrum. Such signals are the result of Doppler displacement of photons when they are colliding with blood cells moving in the blood vessels of the tissue, as is mentioned in for example U.S. Pat. No. 4,590,948 and WO 95/005213.

WO 95/005213 discloses a laser Doppler apparatus by means of which changes in the perfusion in the tissue can be registered non-invasively. Thus, changes in the blood flow of a blood vessel may be monitored. According to WO 95/005213, a specific laser Doppler probe may be arranged outside the skin of the patient and the blood flow below the skin may be monitored.

GB2351197 discloses a similar apparatus for measuring microvascular blood flow transcutaneously, i.e. superficially through the skin. The apparatus includes a device for irradiating a section of a tissue with a monochromatic light from a light source. A device is provided for collecting light backscattered from the irradiated section. A photodetector detects the collected backscattered light. A processor is used for processing the electrical output signals from the photodetector. A calculator calculates a power spectrum of the photo-currents generated in the detection of laser light backscattered from static tissue and Doppler broadened laser light scattered from moving blood cells. An average Doppler frequency shift calculator calculates and records the average Doppler frequency shift. A calculator calculates and records the blood concentration. A detected scattered light intensity meter measures and records the intensity of the detected backscattered light. A blood perfusion (flux) calculator calculates the blood perfusion and a display displays blood perfusion parameters. This apparatus is not suited for interstitial measurements and does not provide tissue therapy or an assessment of the efficiency of such a therapy.

EP1002497 discloses a blood vessel imaging system that includes a measuring light source, which emits a measuring light beam. An optical heterodyne detection system consists of an optical system which splits the measuring light beam into a first light beam travelling to impinge upon an organism and a second light beam travelling not to impinge upon the organism and combines the second light beam with the first beam emanating from the organism into a combined light beam. Because of blood cell movements the frequency in the first beam is shifted with respect to that of the second beam, and a beat component detector can detect the beat (difference frequency) in the combined light beam. A band-pass filter detects, out of the beat component detection signal output from the beat component detector, off-centered components in a frequency band deviated from the center frequency of the beat component detection signal by a predetermined width. An image signal is generated according to whether the off-centered beat signal detected by the band-pass filter is higher or lower than a predetermined threshold level.

U.S. Pat. No. 5,022,757 discloses a heterodyne system and method for sensing a target substance in a medium by directing at least first and second beams of radiation to intersect within the medium and establish one or more sensing volumes. The beams have different frequencies to generate a beat frequency at the sensing volume. A selected optical effect, based on an optical property of the target substance within the sensing volume, on the first and second beams is detected at a selected spectral line. A signal is generated representative of the selected optical effect, such as absorbance or fluorescence, and the portion of the signal which is substantially at the beat frequency is combined with at least one selected value to determine the amount of the target substance.

EP0488614 discloses an apparatus for measuring blood flow that comprises intermittent scanning means for repeatedly carrying out segmental scans each consisting of a rest period and a period during which a segment of a prescribed two-dimensional plane of living tissue is spatially scanned with coherent light. Means are activated for measuring time series data relating to the intensity of scattered light from the tissue during the rest period of each segmental scan. Data relating to the blood within the tissue is calculated from the time series data. The apparatus for measuring blood flow can be provided with a display for displaying the measured data, either alone or as superimposed on an image of the measurement region.

US20030191392 discloses a Doppler guiding catheter that includes a Doppler sensor disposed at a distal end of a flexible shaft. The Doppler sensor can sense a blood flow turbulence level within a chamber of the heart or a blood vessel of the heart. Detecting changes in a blood flow turbulence level is used to assist guiding of the distal end of the flexible shaft. The Doppler sensor may include a piezoelectric sensor or an optical sensor. The sensor readings may be processed to show turbulence through a time domain or frequency domain presentation of velocity. The sensor readings can be used to modulate an audible waveform to indicate turbulence. The guiding catheter may further include steering apparatus enabling deflection of the distal tip.

U.S. Pat. No. 4,608,993 discloses a blood flow measurement device and method. The disclosure relates to systems for measuring blood flow by detecting Doppler shift of ultrasound reflected by blood components moving in a blood vessel. The systems employ electronic techniques for providing accurate tracking of portions of the frequency spectra of Doppler shift signals to determine peak and means velocity and acceleration.

None of the system disclosed in the documents mentioned above are suited for direct interstitial measurements of a liquid flow in a tissue to be exposed to therapy. The systems do not provide tissue therapy or an assessment of the efficiency of such a therapy by a feedback of the flow signal obtained. They serve solely the purpose of determining a blood flow.

Hence, there is a need for a more advantageous device, system and method for determining the effect of therapy in a tumour tissue.

### OBJECTS AND SUMMARY OF THE INVENTION

Accordingly, the present invention preferably seeks to mitigate, alleviate or eliminate one or more of the above-identified deficiencies in the art and disadvantages singly or in any combination and solves, among others, at least partly the above-mentioned issues by providing a device, system, method and use, according to the appended patent claims.

An object of an invention is to use the signals already present in a system for photodynamic or photothermal tumour therapy and diagnosis to determine data on blood flow in the region under diagnosis, and thus a measure for the effectiveness of therapy being performed.

A device for analyzing a liquid flow in a tissue of a human, may comprise a first fibre inserted in a first position of said tissue and connected to a light source such as a laser, a second fibre inserted in a second position of said tissue for receiving light emitted from said first fibre and scattered in said tissue, and a detector arranged for receiving the light from said second fibre for producing an output signal in dependence of the received light. The device comprises a means for analyzing the output signal from said detector and determining a frequency component in the frequency area below about 1 MHz in said output signal.

According to a first aspect of the invention, a device for determining the effect of therapy in a tumour tissue of a human or a mammal under interstitial photodynamic or photothermal therapy is provided. This is done by means of the device, when used, by analyzing a liquid flow in said tumour tissue. The device comprises a first optical fibre for transmitting light between a light source, such as a laser, and said tumour tissue, wherein said first optical fibre has a distal end interstitially inserted into a first position of said tumour tissue and connected to said light source, and a second optical fibre that has a distal end interstitially inserted into a second position, different from said first position, of said tumour tissue for receiving light emitted from said first position at the distal end of said first optical fibre and transmitted therefrom through at least a part of said tissue from said first position and scattered in said tissue to said second position. Further, a proximal detector is arranged for receiving the light received from said tissue and transmitted there from via said second optical fibre for producing an output signal in dependence of the received light. Moreover, the device comprises a means for analyzing the output signal from said detector and configured to determine a frequency component in the frequency area below about 1 MHz in said output signal for determining said liquid flow, and means for determining said effect of said therapy between said first position and said second position as a function of said liquid flow, e.g. anti-proportional to said liquid flow.

The frequency component may have a frequency spectrum from a first frequency, such as about 100 Hz, to a second frequency, such as about 1 MHz. The device may further comprise a means for determining when the intensity of said frequency component is below a predetermined threshold value.

Especially when used in connection with CCD detectors, the device may comprise an oscillator for producing a signal having varying frequency and connected to an input means of said detector for controlling amplification of said detector, and a sensor for sensing a beat signal between said frequency component of the light received by the detector and the amplification frequency. The device further may comprise a means for adjusting the frequency of said oscillator from a first frequency such as about 100 Hz, to a second frequency, such as about 1 MHz. A filter may be connected to the sensor for passing beat signals of a specific passband frequency, such as 50 Hz, said filter being arranged before the sensor. The filter may be embodied as a read out of said CCD detector at video rate (50 Hz). The first frequency may be between 100 Hz and 1 kHz and the second frequency may be about 1 MHz. The second frequency may be dynamically adjusted to a frequency when the beat signal has disappeared.

The above-mentioned effect may be a grade of necrosis or blood coagulation.

According to a further aspect of the invention, there is provided a system for determining the effect of therapy in a tumour tissue of a human or a mammal under interstitial photodynamic or photothermal therapy, by analyzing a liquid flow in said tumour tissue. The system comprises a first optical fibre for transmitting light between a light source, such as a laser, and said tissue, said first optical fibre having a distal end interstitially inserted into a first position of said tumour tissue and connected to said light source, a second optical fibre having a distal end interstitially inserted into a second position, different from said first position, of said tumour tissue for receiving light emitted from said first position at the distal end of said first optical fibre and transmitted therefrom through at least a part of said tissue from said first position and scattered in said tissue to said second position; a first proximal detector arranged for receiving the light received from said tissue and transmitted there from via said second optical fibre for producing a first output signal in dependence of the received light; and a means for analyzing the output signal from said detector and configured to determine a frequency component in the frequency area below about 1 MHz in said output signal for determining said liquid flow, and means for determining said effect of said therapy between said first position and said second position as a function of said liquid flow, e.g. anti-proportional to said liquid flow; at least one additional fibre having a distal end inserted into at least one additional position, different from said first position and said second position, of said tumour tissue, respectively, for receiving light emitted from said first position at the distal end of said first optical fibre and transmitted there from through at least a part of said tissue from said first position and scattered in said tissue to said additional position; a second proximal detector being arranged for receiving the light received from said tissue and transmitted there from via said additional optical fibre for producing an additional output signal in dependence of the received light, whereby the frequency component of the detected light is used to provide three-dimensional information of said flow in said tissue, and thereby three-dimensional information for said effect of said therapy in said tumour tissue as a function of said liquid flow, such as anti-proportional to said liquid flow.

According to an embodiment the system may comprise means for providing a tomographical image for said effect of said therapy in said tumour tissue from said three-dimensional information of said liquid flow.

According to an embodiment the tomographical image may be a 3D image, describing a cell state in said tissue, such as the degree of necrosis in a tumour.

According to yet a further aspect of the invention, a method of determining the effect of therapy in a tumour tissue of a human or a mammal under interstitial photodynamic or photothermal therapy, is provided. The method comprises analyzing a liquid flow in said tumour tissue, by: emitting light through a first fibre interstitially inserted in a first position of said tissue and connected to a light source, such as a laser, receiving light emitted from said first fibre and scattered in said tissue via a second fibre inserted in a second position of said tissue; producing an output signal in dependence of the received light via a detector arranged for receiving the light from said second fibre, and analyzing the output signal from said detector and determining a frequency component in the frequency area below about 1 MHz in said output signal having a frequency from a first frequency, such as about 100 Hz, to a second frequency, such as about 1 MHz.

According to still a further aspect of the invention, a method of determining the effect of therapy in a tumour tissue of a human or a mammal under interstitial photodynamic or photothermal therapy, by measuring a flow in said tumour tissue, is provided. The method comprises interstitially inserting a distal end of a first optical fibre into a first position of said tumour tissue connected to a light source, such as a laser, interstitially inserting a distal end of a second optical fibre into a second position of said tumour tissue for receiving light emitted from said first fibre and scattered in said tissue; arranging a first proximal detector for receiving the light from said second fibre for producing an output signal in dependence of the received light; interstitially inserting at least one additional fibre into at least one additional position of said tissue for receiving light emitted from said first fibre and scattered in said tissue; arranging a second proximal detector for receiving the light from said at least one additional fibre for producing an output signal in dependence of the received light, processing a frequency component of the detected light for providing three-dimensional information of said flow in said tissue, and providing three-dimensional information for said effect of said therapy in said tumour tissue as a function of said liquid flow, such as anti-proportional to said liquid flow.

According to an embodiment, the method may comprise providing a tomographical image for said effect of said therapy in said tumour tissue from said three-dimensional information of said flow. Furthermore, the method may comprise calculating a tomographical image by using tomographical inversion techniques.

In a further aspect, there is provided a use of the above-mentioned device, for analyzing liquid flow in a tissue of a human or a mammal.

### BRIEF DESCRIPTION OF THE DRAWINGS

Other objects, features and advantages of the invention will appear from the following detailed description of embodiments of the invention with reference to the drawings, in which:

FIG. 1 is a schematic view of a previously known device in which the invention may be used;

FIG. 2 is a schematic view similar to FIG. 1 of an embodiment of the invention;

FIG. 3 is a schematic view similar to FIG. 2 of another embodiment of the invention;

FIG. 4 is a block scheme of an analyzing device according to the invention; and

FIG. 5 is a diagram showing the spectral distribution of a Doppler shifted difference signal.

### DETAILED DESCRIPTION OF THE INVENTION

The inventors have surprisingly realized that during the course of photodynamic or photothermal therapy, the blood flow through the tissue changes. This may be used to follow up the course of the treatment by providing a measurement concerning the effect of the therapy performed.

In photo-thermal therapy, the blood in the tissue will coagulate when a certain thermal interaction has been obtained. Such coagulation results in that blood flow ceases and may be an indication that the treatment could stop.

In a similar way, the blood flow changes during photodynamic therapy and this yields information on the progress of the treatment. As the blood flow in a tissue, including tumour tissue, transports oxygen to the tissue, the blood flow is a measure for the amount of oxygen delivered to the tissue. As will be explained below in more detail, oxygen is necessary in the tissue for some photosensitizers to be able to eradicate cells of the tissue upon illumination, whereupon the tissue goes into necrosis. In absence of oxygen, photosensitizers generally are uneffective. On the other hand an absence of oxygen in a tissue leads to necrosis, which is equal with a successful photodynamic therapy of tumour tissue, as the cancer cells are completely eradicated.

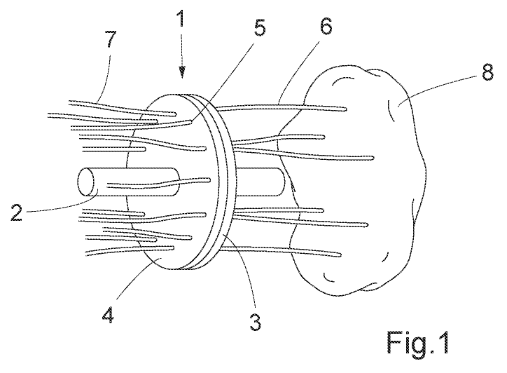

FIG. 1 is a schematic view of a device for interactive photodynamic light therapy (PDT) and/or photo-thermal therapy (PTT) and/or photodynamic diagnosis (PDD) of a site on and/or in a human being or an animal. A plurality or at least two radiation conductors, such as light guides or optical fibres 6 are inserted in a tissue 8, which may be an organ, a tumour or any other tissue. The fibres may be inserted in the tissue 8 in a geometric pattern and interstitially. As shown in FIG. 1, the fibres are arranged in a geometric pattern covering a certain area to be investigated and/or treated. The proximal ends of the fibres 6 arranged at a distance from the tissue are inserted and attached to a plate 3 of a switching means 1, as shown in FIG. 1. The plate 3 is arranged adjacent a second plate 4 and the plates are rotatable in relation to each other around a shaft 2.

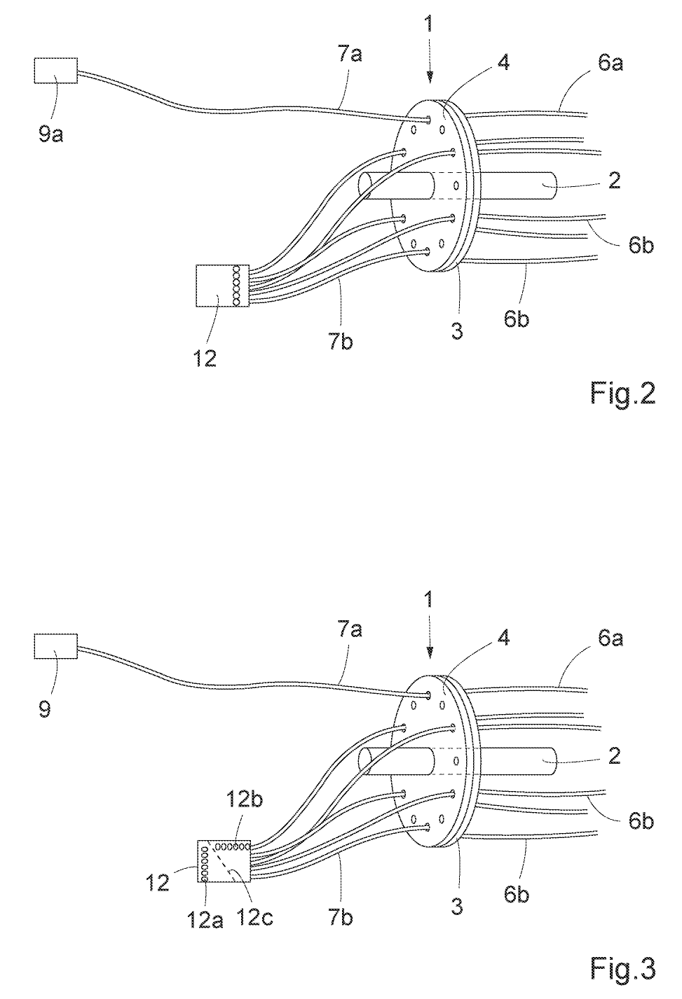

A plurality of or at least two light-guiding fibres 7 are connected to the second plate 4 in openings as shown in FIG. 1. A first fibre 7a, see FIG. 2, is connected to a source of diagnostic light 9a at its proximal end at a distance from plate 4. The diagnostic source may be a laser emitting light at a specific wavelength. Diagnostic light is passed from diagnostic light source 9a via fibre 7a to fibre 6a and to the tissue 8.

The diagnostic light is emitted through the distal end of fibre 6a into the tissue 8 and is scattered inside the tissue. The scattered diagnostic light is picked up by the distal ends of the other fibres 6b and passed to diagnostic sensors arranged in a diagnostic sensor unit 12. Thus, the diagnostic sensor unit 12 is connected to the second plate 4 via light fibres 7b as shown in FIG. 2. The scattered light is passed via fibres 6b and fibres 7b to the diagnostic sensors in unit 12.

By rotating the second plate 4, a new set of fibres 7 are placed opposite the fibres 6, whereupon a new fibre 6 acts as transmitting fibre and the other act as receiving fibres.

The combined reply of the diagnostic sensors in unit 12 (FIG. 2) is evaluated and a diagnostic image of the tissue 8 (FIG. 1) is obtained. Such a diagnostic image may include information about the light flux through the tumour, the auto-fluorescence of the tissue, or a fluorescence signal. Fluorescence is obtained when the tissue is excited with visible or ultraviolet radiation. The last-mentioned fluorescence signal is shifted towards longer wavelength and clearly appears in contrast to the endogenous fluorescence of the tissue. This information is used for localising tumours and for quantifying the size of the uptake of the sensitizer in the tissue. In this way, the correct light dose may be calculated or measured. Microthermistors may be arranged in connection with fibres 6 to measure temperature of the tissue, or temperature may be measured optically through specially prepared fibres.

The tips of the fibres 6 may be prepared with special materials, which yield a temperature-dependent laser-reduced fluorescence signal to allow to measure the temperature of the tissue at the tip of such a specially treated fibre.

However, there is a need to get a direct feedback from photothermal therapy, as measuring the temperature at the tissue during treatment only provides information concerning the amount of energy introduced into the tissue, but not the success of the therapy itself. Furthermore, a more simple system that does not need specially treated optical fibres or microthermistors for temperature measurement would be advantageous, not at least for economical aspects, but also for more reliable systems.

The device in FIGS. 1 and 2 may also be adjusted to a therapy position. In the therapy position, the proximal ends of all fibres may be connected to one or several laser sources, in order to obtain an efficient light radiation of the tissue to be treated.

The light radiation may be infrared light, near-infrared light (NIR) or visible light both in the therapy mode and the diagnostic mode. However, it is to be noted that it is not necessary to use a frequency accurate single mode laser for the Doppler measurements. The reason for this is that a frequency shift occurs around each oscillating mode, and that only the differential frequency is detected. The modes are separated with at least 100 MHz, and therefore the Doppler shift is negligible small in comparison to the modal separation. The differential frequency is immune, although the sum may change. Hence, multimode light sources may be used for certain embodiments.

According to an embodiment of the invention, the diagnostic light transmitted from the distal end of one sender fibre such as fibre 6a, which is interstitially inserted into the tissue 8, to the other receiving fibres 6, is scattered by moving particles, such as moving blood cells in blood vessels or capillaries inside the tissue. The scattering takes place in the tissue from the transmitting fibre towards one the receiving fibre, respectively. During such scattering, a Doppler shift results, in case moving particles provide such a scattering. In case no moving particles are present in the tissue, no Doppler shift results. That means that when moving particles are present, by interference with light scattered by non-moving tissue, such as the walls of a blood vessel, or non-moving tumour tissue, interference signals are produced which are the sum and the difference, respectively, between the Doppler shifted light and the non-shifted light. For instance, if the blood cells that are moving in a tissue with a speed of 4 mm/sec, the Doppler shift will be in the range of 104 Hz for near infrared light. It is realised that the sum signal cannot normally be detected. However, the difference signal may be detected by suitable detectors.

Since blood perfusion in tissue has no preferred direction and since the light is scattered several times at the passage from the sender fibre to the receiver fibres, Doppler shift interference signals will be obtained having a statistical frequency distribution. From the recorded frequency distribution the blood perfusion may be calculated as described, e.g. in WO 95/005213. However, WO 95/005213 is based on a distal placement of the optical fibres in relation to tissue, so that these Doppler measurements were based on light backscattered from the tissue, in contrast to the present embodiment. WO 95/005213 is based on superficial measurements.

However, when blood coagulates, the movement of blood cells in the tissue ceases and there will be no resulting Doppler shift and interference. The inventors have realized that this may be interpreted as an indication that the treatment has been successful. Therefore the disappearance of such Doppler interference signals may be used as an indication to interrupt the treatment in progress. Hence, a temperature measurement of the photothermal temperature at the location in the tissue under photothermal treatment is no longer necessary and may be omitted according to this embodiment of the invention.

In a similar way, the blood flow changes during photodynamic therapy and this yields information on the progress of the treatment, for instance by tissue being locally killed, which is also called that the tissue is going into necrosis. Here, similarly the effect of therapy is determined. Necrosis may occur at a delay, so the blood-flow reaction may be different during the treatment but still characteristic. The therapy could be more successful the smaller the Doppler shift and the interference is, i.e. the effect of therapy could be anti-proportional to this measurement.

A photosensitizing agent may be ALA solution, phtalocyanines, chlorines etc. These agents act in the following manner for therapy of the tumour. ALA or aminolevulinic acid is a precursor photosensitizer. When applied, ALA is converted into protoporphyrin IX (PPIX), which is a photosensitizer. The process is more efficient in tumour cells. By applying a laser beam, at a wavelength of approximately 635 nm, on the tumour, the PPIX is excited and the excitation is transferred to oxygen in the cells, which is promoted from its triplet ground state to its excited singlet state. The singlet state oxygen will fatally damage the cells and within a few days the cells in the affected area are dead. Phtalocynanines are a second generation of photosensitizers with improved pharmaceutical profiles. They have a strong absorption in the red region in which tissue is rather transparent making them suitable for PDT. Chlorines are also a second generation photosensitizer suitable for PDT. Other sensitizers are Foscan and Tookad. The action of Tookad is mainly a closedown of the tumour vasculature.

In the above-described previously known diagnosis method, CCD detectors are frequently used for spectral evaluation of the light in the receiving fibres. Such detectors do not normally have a sufficiently fast read-out response for detecting the Doppler-induced interference. According to some embodiments, a heterodyne detection technique, as will be further described below, may be used.

In a first embodiment of the invention, separate detectors are used for the spectral evaluation and the Doppler interference evaluation. Such an embodiment is schematically shown in FIG. 3. Detector unit 12 may comprise two detectors for each receiving fibre, one 12a for the spectral evaluation and one 12b for the Doppler difference evaluation. A semitransparent mirror 12c is arranged for reflecting a portion of the light to the detectors 12b for Doppler difference evaluation.

The mirror 12c may alternatively be a non-transparent mirror, which is pivoted between the position shown in FIG. 3, in which the light is reflected to the Doppler sensors 12b, and a position in which the light passes straight ahead to the spectral detectors 12a. Other methods for diverting the light to the Doppler detector 12b or the spectral detector 12a may be used as well, such as using rotating or displaceable disks, as is shown in WO 03/041575. Alternatively, an optical fibre may be provided with a bifurcation towards the photodetector, e.g. with a distribution of 80% towards a CCD and 20% towards a fast photodiode.

The detector for Doppler evaluation may be a photo diode or other detector having a response characteristic, which makes it possible to detect frequency components in the range from 100 Hz to 10 MHz.

The amplitude of these frequency components has a relationship to the number of cells moving at a speed corresponding to said frequency components, see the diagram shown in FIG. 5. If the intensity A of any frequency component within said frequency range is above a predetermined threshold value, it is an indication of the presence of moving cells. If the intensity is below said threshold value, it is supposed that no moving cells are present, which is an indication of tissue necrosis or blood coagulation, i.e. how effective therapy is progressing.

In certain applications, it is possible to use the same detector for photodynamic diagnosis and Doppler evaluation without any modification. In this case, no mirror or other switching means is required.

Thus, according to another embodiment of the invention the same detector is used for both spectral evaluation and Doppler difference evaluation, which detector is a CCD sensor. If the amplification of the detector is modulated or adjusted with a frequency equal to the Doppler difference frequency, either a cancellation or amplification of the Doppler interference frequency is obtained depending on the phase of the amplification variation and the Doppler difference signal. Moreover, if the amplification frequency and the Doppler difference frequency are close to each other, a beat or variation with a slow frequency will be obtained, which may be detected. Thus, by adjusting the amplification of the detector by a frequency, which is swept over a range from 102 Hz to 106 Hz, and observing the beat frequency, the Doppler difference frequency in a frequency pass band can be obtained.

The amplification may be adjusted by a certain factor and the frequency starts at 100 Hz and the output from the detector is monitored. If there is a variation in the response from the detector, this is an indication of a Doppler difference signal. This difference signal is measured e.g. at 50 Hz. Then the amplification frequency is increased in steps by e.g. 100 Hz and a new evaluation is made for each step.

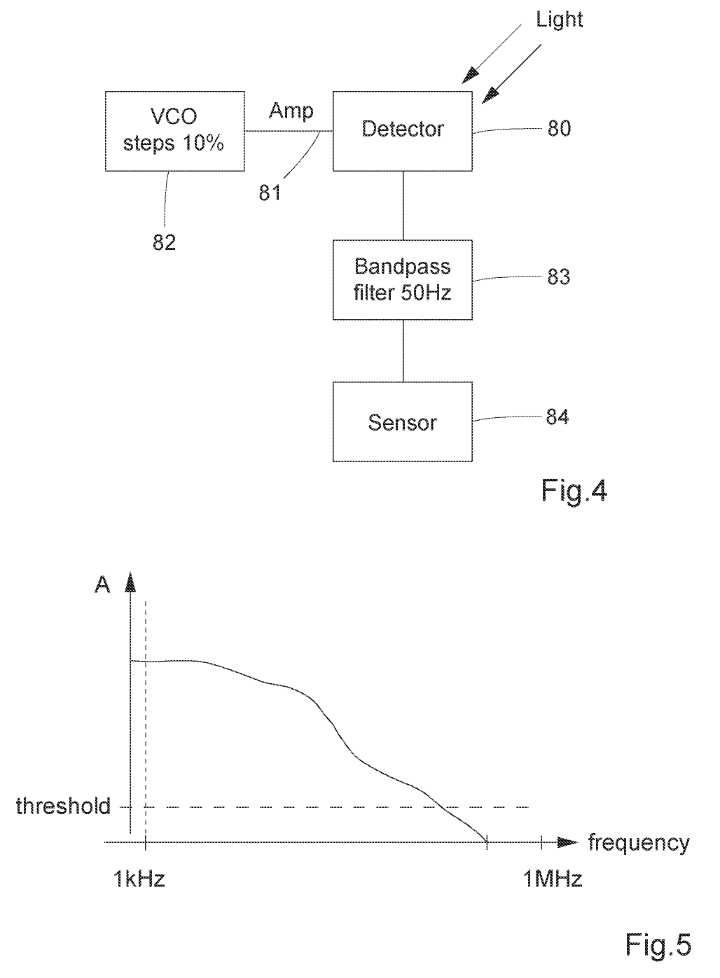

FIG. 4 is a block diagram of this embodiment. The detector 80 has a specific amplification, which is controlled by a voltage on an amplification contact 81 of the detector. A voltage-controlled oscillator 82 is connected to the amplification contact 81. The voltage-controlled oscillator is arranged to emit an voltage of a specific frequency and amplitude. The amplitude is selected so that the amplification of the detector varies appropriately. The frequency is measured in steps to cover the full Doppler shift range.

Because of the amplification variation, a beat signal is produced. By analysing the beat signal at the frequency of e.g. 50 Hz, the Doppler difference signal can be obtained. A bandpass filter 83 having a centre frequency of 50 Hz is connected to the output of detector 80. The signal passing the bandpass filter is analysed by a sensor 84. When the oscillation signal is swept, the sensor determines the distribution of amplitudes of the Doppler difference signal. By using the frequency 50 Hz, it is possible to read out at the video frequency. We note that a EED is an imaging detector that input signals from many fibres can be processed simultaneously.

FIG. 5 shows a typical diagram obtained from the unit 12. As appears from FIG. 5, there is no signal above a specific frequency. This means that the sweeping of the frequency can be stopped when this upper frequency has been obtained. This upper frequency is indicative of the maximum velocity of the blood cells in the vessels or capillaries of the tissue. This maximum velocity may typically be 3 cm/sec for capillaries resulting in a frequency of about 3*104 Hz for red or near-IR light.

If the amplitude of the Doppler signal passes below a predetermined threshold, as indicated in FIG. 5, this may be interpreted as an indication that there is substantially no moving blood cells. Then, the treatment may be interrupted in thermal therapy, or in, e.g. Tookad photodynamic therapy.

The analyzing of the flow of blood cells normally cannot take place at the same time as a treatment, since during the treatment, all fibres are normally used for transmitting high power laser light into the tissue. However, one of the fibres may be left for the analyzing according to the present invention. Alternatively, the device is regularly switched between a treatment mode and an analyzing mode.

By having a transmitting fibre and at least two receiving fibres, 3D-measurements are obtainable. In this manner the blood perfusion is provided three-dimensionally in the tissue under treatment, which is of interest for controlling the therapy. In this manner, a system is provided, where the therapy is locally adapted to the present flow distribution. On the other hand, regions of the tissue may in this manner be identified, where therapeutic radiation may be stopped, preventing disadvantageous patient damage due to over-radiation.

Furthermore, by using a plurality of such three-dimensional measurements, a system is provided that gives a tomographic image of the effect of therapy on the tissue. Such tomographic images may be provided by known calculation methods, e.g. by using the system shown in FIG. 1. Here, light from a radiation source is transmitted through one particular radiation conductor 7 via the discs 4,3 into the tissue 8, through one of the radiation conductors 6, which will function as a transmitter into the tumour, and the other five radiation conductors 6 in the tumour will act as receivers and collect the diffuse flux of light reaching them. The light collected is again conducted via the discs 3,4, to a radiation sensor and five different light characteristics may be recorded, e.g. on a detector array. When the turnable disc 4 is turned by 60 degrees, the next radiation conductor 6 to the patient will get the role as transmitter, and the five others become the receivers for a new light distribution. After four further turns of the turnable disc 4, each by 60 degrees to the following radiation conductor 6 in the patient, light flux data for all remaining combinations of transmitters/receivers have been recorded. Thus, in total 6×5=30 measurement values are obtained and can be used as input data for a tomographic modelling of the effect of therapy on the tissue in the different parts of the tumour during the course of the treatment.

Moreover, detailed information concerning the development of therapy overtime time is obtainable, e.g. displayable as a trend curve over time for visualizing the progress of the photodynamic or photothermal therapy.

Also, tomographic images obtained by conventional light flux measurements may be used or displayed in combination with tomographic information obtained by liquid flow Doppler measurements, obtained as described above in detail. For instance liquid flow information may be overlayed with conventional tomographic image information in order to obtain a more reliable diagnosis of the progress of tumour therapy. This may lead to increased patient safety by minimizing radiation dosages or medicaments, such as sensitizers, applied or administered to the patient.

Herein above, specific embodiments of the invention have been described with reference to the drawings. However, the different features may be used in different combinations than explicitly disclosed in the embodiments. The invention is only limited by the appended patent claims.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A device for determining an effect of interstitial photodynamic or photothermal therapy on a tumour tissue of a human or a mammal, configured to determine said effect during said therapy in progress by interstitial measurement of a liquid flow in said tumour tissue, said device comprising
a first optical fibre for transmitting light between a light source, such as a laser, and said tissue, said first optical fibre having a distal end interstitially inserted into a first position of said tumour tissue and connected to said light source,
a second optical fibre having a distal end interstitially inserted into a second position, different from said first position, of said tumour tissue for receiving light emitted from said first position at the distal end of said first optical fibre and transmitted therefrom through at least a part of said tissue from said first position and scattered in said tissue to said second position;
a proximal detector unit arranged for receiving the light received from said tissue and transmitted therefrom via said second optical fibre for producing an output signal in dependence of the received light; and
a unit for analyzing the output signal from said detector and configured to determine a frequency component in the frequency area below about 1 MHz in said output signal for determining said liquid flow, and
a unit for determining said effect of said therapy between said first position and said second position as a function of said liquid flow, such as anti-proportional to said liquid flow.

2. The device of claim 1, wherein said frequency component has a frequency spectrum from a first frequency, such as about 100 Hz, to a second frequency, such as about 1 MHz.

3. The device of claim 1, further comprising
a unit for determining when the intensity of said frequency component is below a predetermined threshold value.

4. The device of claim 1, further comprising
an oscillator for producing a signal having varying frequency and connected to an input means of said detector for controlling amplification of said detector, being optionally a CCD detector, and
a sensor for sensing a beat signal between said frequency component of the light received by the detector and the amplification frequency.

5. The device of claim 4, further comprising a means for adjusting the frequency of said oscillator from a first frequency such as about 100 Hz, to a second frequency, such as about 1 MHz.

6. The device of claim 4, wherein a filter connected to the sensor for passing beat signals of a specific passband frequency, such as 50 Hz, said filter being arranged before the sensor, whereby the filter is optionally embodied as a read out of said CCD detector at video rate (50 Hz).

7. The device of claim 2, wherein the first frequency is between 100 Hz and 1 kHz and the second frequency is about 1 MHz.

8. The device of claim 4, wherein the device is configured to dynamically adjust the second frequency to a frequency when the beat signal has disappeared.

9. The device according to claim 1, wherein said effect is a grade of necrosis or blood coagulation.

10. The device according to claim 1, wherein said therapy is photothermal therapy and said liquid flow is detected by means of a Doppler measurement providing a measurement for a movement of blood cells between said first position and said second position.

11. The device according to claim 1, wherein said light source is a therapeutic light source providing said therapy, whereby the effect of the therapy is determined concurrently with said therapy in progress.

12. The device according to claim 1, wherein said device is adapted to regularly switch between a treatment mode for providing said therapy and an analyzing mode for determining said effect of said therapy, such that said therapy is interactively controlled by said determined effect.

13. The device according to claim 12, wherein said device is comprised in a system for interactive photodynamic light therapy (PDT) and/or photo-thermal therapy (PTT) and/or photodynamic diagnosis (PDD).

14. The device according to claim 1, wherein said detector unit comprises a separate first detector for spectral evaluation of the light received by said second fiber, and a second detector for a Doppler interference evaluation providing said measurement of said liquid flow.

15. The device according to claim 14, wherein a mirror is arranged for reflecting a portion of the light to the second detector.

16. The device according to claim 14, wherein rotatable or displaceable disks are configured to direct light between said first detector and said second detector.

17. A system for determining an effect of interstitial photodynamic or photothermal therapy on a tumour tissue of a human or a mammal, configured to determine said effect during said therapy in progress by interstitial measurement of a liquid flow in said tumour tissue, said system comprising
a first optical fibre for transmitting light between a light source, such as a laser, and said tissue, said first optical fibre having a distal end interstitially inserted into a first position of said tumour tissue and connected to said light source,
a second optical fibre having a distal end interstitially inserted into a second position, different from said first position, of said tumour tissue for receiving light emitted from said first position at the distal end of said first optical fibre and transmitted therefrom through at least a part of said tissue from said first position and scattered in said tissue to said second position;
a first proximal detector unit arranged for receiving the light received from said tissue and transmitted therefrom via said second optical fibre for producing a first output signal in dependence of the received light; and
a unit for analyzing the output signal from said detector and configured to determine a frequency component in the frequency area below about 1 MHz in said output signal for determining said liquid flow, and
a unit for determining said effect of said therapy between said first position and said second position as a function of said liquid flow, such as a anti-proportional to said liquid flow; and
at least one additional optical fibre having a distal end inserted into at least one additional position of said tumour tissue, different from said first position and said second position, of said tumour tissue, respectively, for receiving light emitted from said first position at the distal end of said first optical fibre and transmitted therefrom through at least a part of said tissue from said first position and scattered in said tissue to said additional position;
a second proximal detector being arranged for receiving the light received from said tissue and transmitted therefrom via said additional optical fibre for producing an additional output signal in dependence of the received light,
whereby the frequency component of the detected light is used to provide three-dimensional information of said flow in said tissue, and thereby three-dimensional information for said effect of said therapy in said tumour tissue as a function of said liquid flow, such as anti-proportional to said liquid flow.

18. The system according to claim 17, further comprising means for providing a tomographical image for said effect of said therapy in said tumour tissue from said three-dimensional information of said flow.

19. The system according to claim 18, wherein said frequency component has a frequency spectrum from a first frequency, such as about 100 Hz, to a second frequency, such as about 1 MHz.

20. The system according to claim 18, further comprising a means for determining when the intensity of said frequency component is below a predetermined threshold value.

21. The system according to claim 18, further comprising an oscillator for producing a signal having varying frequency and connected to an input means of said first detector and said second detector for controlling amplification of said first detector and said second detector, being optionally a CCD detector, and
a sensor for sensing a beat signal between said frequency component of the light received by said first detector and said second detector and the amplification frequency.

22. The system according to claim 21, further comprising a means for adjusting the frequency of said oscillator from a first frequency such as about 100 Hz, to a second frequency, such as about 1 MHz.

23. The system of claim 21, further comprising a filter connected to said sensor for passing beat signals of a specific passband frequency, such as 50 Hz, wherein said filter is arranged before said sensor, whereby said filter is optionally embodied as a read out of said CCD detector at video rate (50 Hz).

24. The system of claim 19, wherein said first frequency is between 100 Hz and 1 kHz and said second frequency is about 1 MHz.

25. The system of claim 21, wherein said second frequency is dynamically adjusted to a frequency when said beat signal has disappeared.

26. The system according to claim 18, wherein said tomographical image is a 3D image, describing a cell state in said tissue, such as the degree of necrosis in a tumour.

27. The system according to claim 17, wherein said system is configured to locally adapt said therapy to a present three dimensional flow distribution.

28. The system according to claim 17, wherein said system further is configured to overlay tomographic images obtained by conventional light flux measurements with tomographic information obtained by liquid flow Doppler measurements.

29. A method of determining an effect of interstitial photodynamic or photothermal therapy on a tumour tissue of a human or a mammal, wherein said effect is determined during said therapy in progress, said method comprising
analyzing a liquid flow in said tumour tissue, by:
emitting light through a first fibre interstitially inserted in a first position of said tissue and connected to a light source, such as a laser,
receiving light emitted from said first fibre and scattered in said tissue via a second fibre inserted in a second position of said tissue;
producing an output signal in dependence of the received light via a detector arranged for receiving the light from said second fibre, and
analyzing the output signal from said detector and determining a frequency component in the frequency area below about 1 MHz in said output signal having a frequency from a first frequency, such as about 100 Hz, to a second frequency, such as about 1 MHz.

30. A method of circumventing a temperature measurement of a photothermal temperature at a location in tissue under photothermal treatment comprising analyzing a liquid flow in said tumour tissue, said method comprising
emitting light through a first fibre interstitially inserted in a first position of said tissue and connected to a light source, such as a laser,
receiving light emitted from said first fibre and scattered in said tissue via a second fibre inserted in a second position of said tissue;
producing an output signal in dependence of the received light via a detector arranged for receiving the light from said second fibre, and
analyzing the output signal from said detector and determining a frequency component in the frequency area below about 1 MHz in said output signal having a frequency from a first frequency, such as about 100 Hz, to a second frequency, such as about 1 MHz.

31. A method of determining an effect of interstitial photodynamic or photothermal therapy on a tumour tissue of a human or a mammal, wherein said effect is determined during said therapy in progress by measuring a flow in said tumour tissue, said method comprising
interstitially inserting a distal end of a first optical fibre into a first position of said tumour tissue connected to a light source, such as a laser,
interstitially inserting a distal end of a second optical fibre into a second position of said tumour tissue for receiving light emitted from said first fibre and scattered in said tissue;
arranging a first proximal detector for receiving the light from said second fibre for producing an output signal in dependence of the received light;
interstitially inserting at least one additional fibre into at least one additional position of said tissue for receiving light emitted from said first fibre and scattered in said tissue;
arranging a second proximal detector for receiving the light from said at least one additional fibre for producing an output signal in dependence of the received light,
processing a frequency component of the detected light for providing three-dimensional information of said flow in said tissue, and providing three-dimensional information for said effect of said therapy in said tumour tissue as a function of said liquid flow, such as anti-proportional to said liquid flow.

32. The method according to claim 31, further comprising providing a tomographical image for said effect of said therapy in said tumour tissue from said three-dimensional information of said flow.

33. The method according to claim 32, comprising calculating said tomographical image by using tomographical inversion techniques.

34. A method of circumventing a temperature measurement of a photothermal temperature at a location in tissue under photothermal treatment, said method comprising
measuring a flow in said tumour tissue, said method comprising
interstitially inserting a distal end of a first optical fibre into a first position of said tumour tissue connected to a light source, such as a laser,
interstitially inserting a distal end of a second optical fibre into a second position of said tumour tissue for receiving light emitted from said first fibre and scattered in said tissue;
arranging a first proximal detector for receiving the light from said second fibre for producing an output signal in dependence of the received light;
interstitially inserting at least one additional fibre into at least one additional position of said tissue for receiving light emitted from said first fibre and scattered in said tissue;
arranging a second proximal detector for receiving the light from said at least one additional fibre for producing an output signal in dependence of the received light,
processing a frequency component of the detected light for providing three-dimensional information of said flow in said tissue, and providing three-dimensional information for said effect of said therapy in said tumour tissue as a function of said liquid flow, such as anti-proportional to said liquid flow.
